(19) United States
(12) Patent Application Publication
(10) Pub. No.: US 2018/0121080 A1
(43) Pub. Date: May 3, 2018

(54) Display Having Position Sensitive Keyboard

(71) Applicant: DRAEGER MEDICAL SYSTEMS, INC., Andover, MA (US)

(72) Inventors: Timothy Joseph COONAHAN, Sterling, MA (US); Juan Pablo ESLAVA, Groton, MA (US)

(21) Appl. No.: 15/564,171

(22) PCT Filed: Sep. 1, 2015
(86) PCT No.: PCT/US2015/047938

§ 371 Date: Oct. 3, 2017

(51) Int. Cl. G06F 3/0488; G06F 3/041; G16H 40/63

(52) U.S. Cl. CPC G06F 3/04886; G16H 40/63; G06F 3/0416; G06F 3/0412

(57) ABSTRACT

A first view is rendered in a graphical user interface of a touch screen display, when a front face of the display, on which the graphical user interface is rendered, is positioned within a predefined angle range relative to vertical. The first view does not include an interactive keyboard. Thereafter, it is detected, by a position sensor that an angle of the front face of the display is beyond the predefined angle range. The graphical user interface then, in response to the detection of movement, automatically renders a second view that includes an interactive keyboard. Related apparatus, systems, techniques and articles are also described.

### TECHNICAL FIELD

The subject matter described herein relates to displays having position sensors that are used to selectively render or project keyboards for use in connection with such displays.

### BACKGROUND

Data entry plays an important component with most workflows. For example, with a clinical workflow, data entry is especially important to update or otherwise change the manner of care for a particular patient. For example, caregivers can use data entry devices such as keyboards for logging which medication has been given to a patient at any given time and/or to change one or more operating parameters of a medical device. Keyboards, in particular, pose problems in a clinical setting as they can take up much needed space within a particular area and/or they can require sterilization after each patient use. Alternatively, sterilization covers can be used, however, such covers often reduce the usability and effectiveness of keyboards.

### SUMMARY

In a first aspect, a first view is rendered in a graphical user interface of a touch screen display, when a front face of the display, on which the graphical user interface is rendered, is positioned within a predefined angle range relative to vertical. The first view does not include an interactive keyboard. Thereafter, it is detected, by a position sensor that an angle of the front face of the display is beyond the predefined angle range. The graphical user interface then, in response to the detection of movement, automatically renders a second view that includes an interactive keyboard.

In some implementations, at least one of the first view or the second view comprises patient medical data (e.g., dynamically changing patient medical data derived from physiological sensors, etc.) and the display forms part of a patient monitor.

The detecting can include detecting that the angle of the front face of the display has exceeded the predefined angle range relative to vertical beyond a predefined amount of time.

The second view can encapsulate at least a portion of the first view. In some variations, the interactive keyboard can obscure at least a portion of the first view. In still other variations, the second view can solely include the interactive keyboard (e.g., no other data other than an input box showing the entered keys can be displayed, etc.). Further, some variations can provide that the keyboard does not obscure any portion of the first view in the second view.

The display can take many forms. In some variations, the display can be secured to a first end of a mounting arm that is, in turn, configured to be secured to a surface at a second end of the mounting arm. The position sensor can be within the mounting arm. The mounting arm can include a joint at the first end such that the position sensor detects relative motion between the joint and the display.

In some implementations, the position sensor can be within a housing of the display.

The position sensor can take many forms such as an accelerometer, an angular position sensor, a gyro sensor, a linear position sensor, a magnetic/Hall Effect sensor, a mechanical switch, and/or an optical switch.

A proximity sensor can also be incorporated to determine that a user of the display is no longer within a predefined distance from the display. In response to such determination, the interactive keyboard can be removed from the second view.

In an interrelated aspect, a view is rendered in a graphical user interface of a display having an associated and movable keyboard tray when the keyboard tray is in a first position. It is later detected, by a position sensor (which may be in the keyboard tray), that the keyboard tray has change in position relative to the first position beyond a predefined amount. In response to the detection, projection of a virtual keyboard on a surface of the keyboard tray is then automatically initiated.

The change in position of the keyboard tray relative to the first position can be based on at least one of lateral motion or angular motion of the keyboard tray.

The first view can include patient medical data (e.g., dynamically changing patient medical data derived from physiological sensors, etc.) and the display forms part of a patient monitor.

The detecting can include detecting that an angle of the keyboard tray has exceeded a predefined angle range. The detecting can alternatively or additionally include detecting that a lateral position of the keyboard tray has exceeded a predefined distance relative to the first position.

The detecting can include detecting that the change in position from the first position has exceeded a predefined amount of time.

The position sensor within or used in connection with the keyboard tray can include one or more of an accelerometer, an angular position sensor, a gyro sensor, a linear position sensor, and a magnetic/Hall Effect sensor, a mechanical switch, and an optical switch.

It can be determined, by a proximity sensor, that a user of the display is no longer within a predefined distance from the display. In response to such determination, projection of the virtual keyboard can be ceased (i.e., terminated, etc.).

In still a further interrelated aspect, a graphical user interface of a touch screen display renders a first view when a front face of the display, on which the graphical user interface is rendered, is positioned vertically or within a predefined angle range relative to vertical. This first view does not include a user input area (e.g., keyboard, touchscreen interface, other graphical user interface input elements, etc.). Thereafter, it can be detected, by at least one position sensor within or adjacent to the display, that an angle of the front face of the display is beyond the predefined angle range. A second view is then automatically rendered in the graphical user interface that includes a user input area in response to the detecting.

In yet another interrelated aspect, a system can include a display having (i) a housing, (ii) at least one programmable data processor, (iii) memory, and also include a mounting arm having a position sensor that is secured, on a first end, to the housing, and is configured to be secured, on a second end, to a surface . The memory can store instructions which, when executed by the at least one programmable data processor, implement aspects of the methods described herein.

The mounting arm, with such system, can include a joint at the first end such that the position sensor detects relative motion between the joint and the display.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g. the Internet, a wireless wide area network, a local area network, a wide area network, a wired network, or the like), via a direct connection between one or more of the multiple computing systems, etc.

The subject matter described herein provides many technical advantages. For example, the current subject matter enables improved (e.g., clinical, etc.) workflows by providing a keyboard on demand through natural user actions. Furthermore, with regard to clinical settings, the current subject matter is also advantageous in that keyboard functionality is provided in an arrangement that is easy to disinfect.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1A is a diagram illustrating a display in a first position in which a keyboard is not displayed in a first view;

FIG. 1B is a diagram illustrating the display of FIG. 1A in a second position in which an interactive keyboard is displayed in a second view;

FIG. 2 is a diagram illustrating the first view of FIG. 1A;

FIG. 3 is a diagram illustrating a first variation of the second view of FIG. 1B including an interactive keyboard;

FIG. 4 is a diagram illustrating a second variation of the second view of FIG. 1B including an interactive keyboard and a user input area in a first position;

FIG. 5 is a diagram illustrating a third variation of the second view of FIG. 1B including an interactive keyboard and a user input area in a second position;

FIG. 6 is a diagram illustrating a fourth variation of the second view of FIG. 1B including an interactive keyboard and a user input area in a second position and also including a submenu region;

FIG. 7A is a diagram illustrating a display having an accessory;

FIG. 7B is a diagram illustrating the display of FIG. 4A in which a virtual keyboard is illustrated on the accessory;

FIG. 8 is a process flow diagram for selectively rendering an interactive keyboard in a display based on movement of the display;

FIG. 9 is a process flow diagram for selectively projecting an interactive keyboard on an accessory of a display in response to movement of the accessory.

Like reference symbols in the various drawings indicate like elements.

### DETAILED DESCRIPTION

The current subject matter is directed to displays (i.e., an electronic screen such as a monitor for conveying visual and/or audio information, etc.) in which a keyboard can be automatically activated/made available in response to movement of the display and/or an accessory associated with the display. As will be described in further detail below, in some variations, the movement (e.g., translational, rotational, angular, a combination of the foregoing, etc.) of a touch screen display from a first position to a second position will cause a view rendered in the graphical user interface of the display to include a user input area (e.g., an interactive keyboard, a touchpad interface, etc.). In other variations, the display can include a keyboard tray which, when moved, causes a virtual keyboard to be projected thereon. Both arrangements are provided in a manner that provides enhanced usability (especially as part of clinical workflows) while, at the same time, allows for easy disinfection of the equipment.

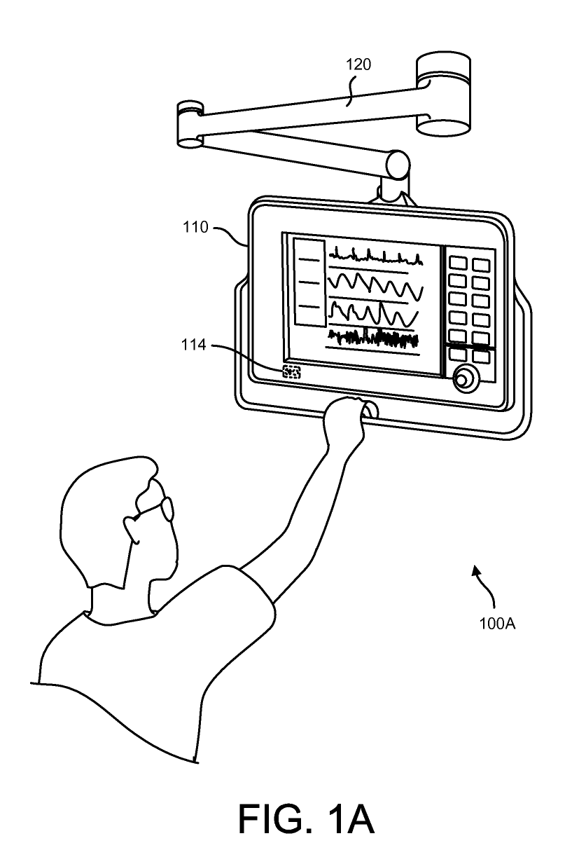

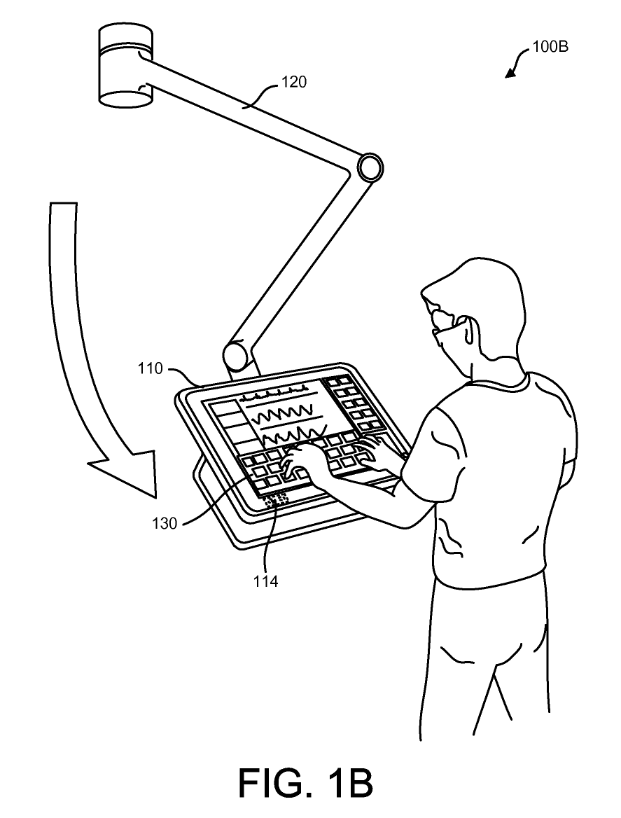

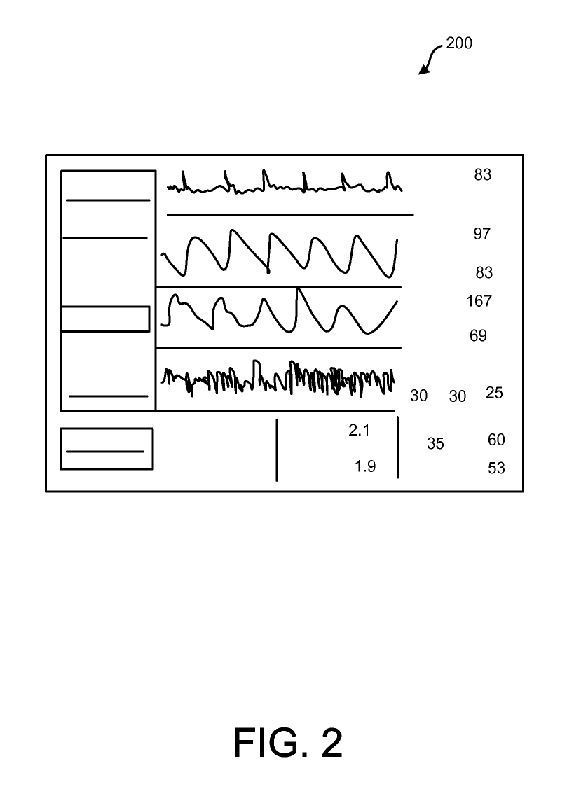

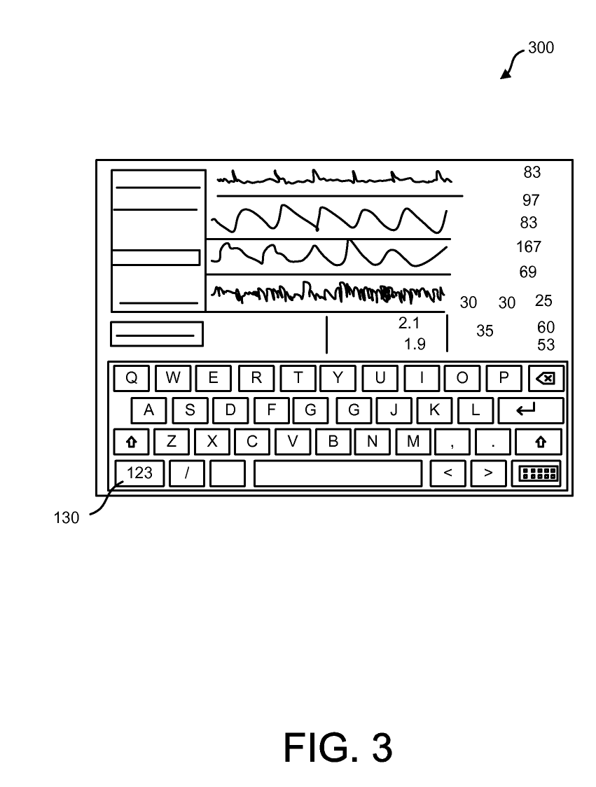

FIGS. 1A and 1B are diagrams 100A and 100B showing a display 110 that includes a graphical user interface that illustrates a first view illustrating, for example, patient medical data (such as that in diagram 200 of FIG. 2) and a second view illustrating, for example, patient medical data (such as that in diagram 300 of FIG. 3) in FIG. 1B that includes an interactive keyboard 130. The patient medical data can, for example, be visualizations or other data characterizing measurements from physiological sensors coupled to a patient, historical patient treatment information, and the like. It will be appreciated that unless specified otherwise, the current subject matter is applicable to any type of display type as well as to various graphical user interface renderings (and not just applicable to healthcare or medical applications). Furthermore, it is appreciated that the keyboards are provided as examples of user input areas in which a user can input data and that other interfaces such as touchpad interfaces and the like can be utilized.

The display 110 can, for example, include a mounting arm 120 which can be affixed to a wall, ceiling, or other surface that can allow for the selective movement and securing of the display 110 by a user. In some variations, the mounting arm 120 is an articulating arm. For example, a user can move the display 110 from a first position in which a front face of the display is substantially perpendicular relative to the floor (as in FIG. 1A) to a second position in which the display is secured in a position that is angled relative to the floor (as in FIG. 1B). The front face of the display, in this regard, refers to the side of the display having a screen on which various graphical views are rendered. Such an angled position allows a user to more readily interact with the touch screen of the display 110 using both hands (i.e., the display 110 need not be held by the user, etc.). Furthermore, it will be appreciated that the mounting arm 120 is advantageous in that it can provide sufficient resistance so that the position of the display 110 does not inadvertently move which can, in turn, cause the interactive keyboard 130 to unintentionally be added and/or removed from a particular view.

The display 110 can be a touch-screen display having an input device layered on top of the screen by which a user can give input through simple or multi-touch gestures by touching the screen with a special stylus/pen and-or one or more fingers. In some cases, touch-screen display can comprise a touchless screen display in which the input device need not require actual physical touch in order to receive user-generated input but rather, the user need only hover his or her fingers and/or stylus pen near the top of the screen without actual contact.

As noted in FIG. 2 and FIGS. 4-6, in the first position, the first view of the display 110 does not include a keyboard and, in the second positon, the second view of the display 110 includes an interactive keyboard 130 which may or may not obscure information presented in the first view. To effect such a change without user intervention (other than by physically moving the display 110), the display can comprise at least one position sensor 114 that can detect movement of the display 110 and/or a change in orientation of the display 110 in relation to one or more objects and generate corresponding signals characterizing same. The dashed lines for position sensor 114 indicate that the position sensor can, in some variations, be a component internal to the display 110 (i.e., enclosed within a main housing of the display). The position sensor 114 can be at any position within or coupled to the display 110 such that movement of the display 110 can be characterized or otherwise detected. In some variations, the position sensor 114 can be within the mounting arm 120 and it can detect movement of at least a portion of the mounting arm 120 and/or a change in orientation of the mounting arm in relation to the display 110. Further, while reference is made to a single position sensor 114 it can be appreciated that there can be two or more position sensors 114 that generate signals indicative of position (e.g., absolute position or orientation, relative movement or orientation, etc.) which can act in concert or be wholly separate. Example position sensors 114 include one or more of: accelerometers, angular position sensors, gyro sensors, linear position sensors, magnetic/Hall Effect sensors, and the like.

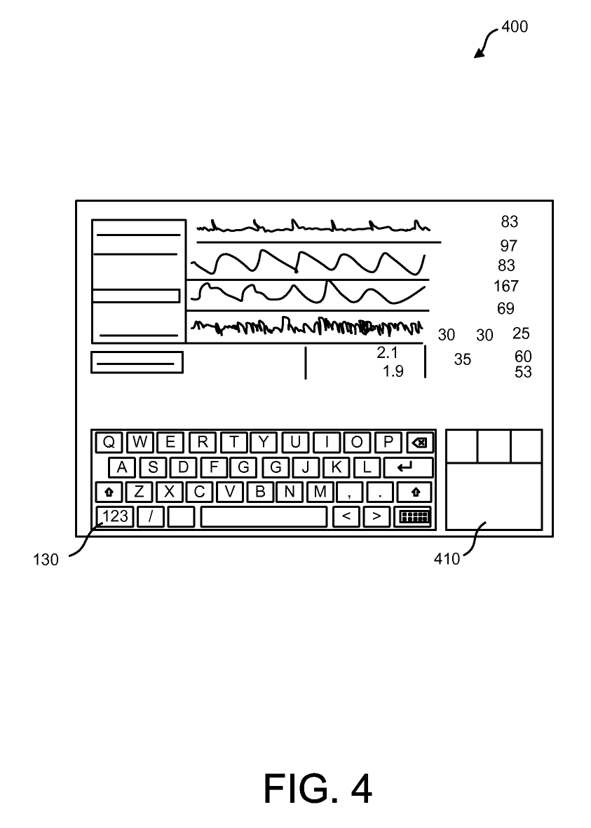

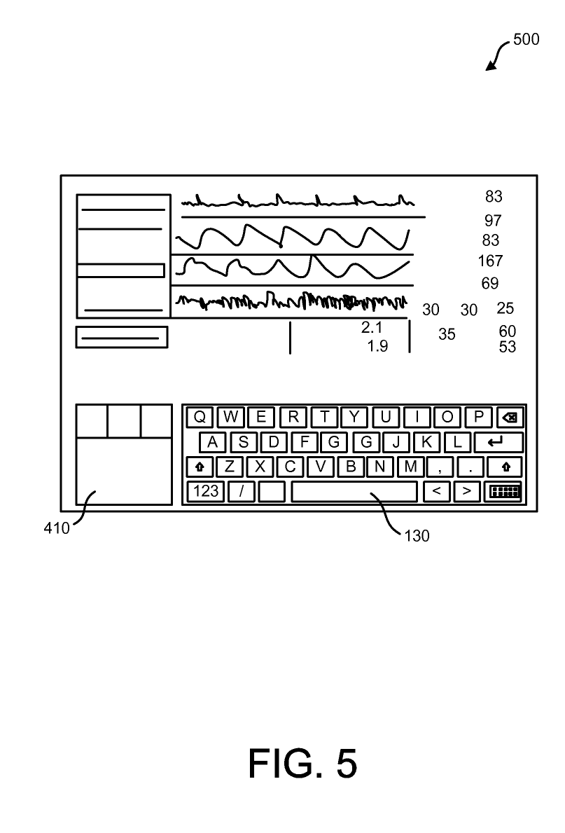

In some variations, as illustrated in diagrams 400, 500 of FIGS. 4 and 5, the display 110 can, for example, include a view having a graphical user interface option providing an interactive keyboard 130 and a touchpad interface 410 for instances in which this type of interface combination is desirable and/or necessary. The touchpad interface 410 can enable familiar mouse gestures to be used such as right clicking for menus, tapping for acknowledgment, and navigating graphical user interfaces that were designed for a mouse. This touchpad interface 410 can be left handed or right handed in relation to the interactive keyboard 130 as respectively shown in FIGS. 4 and 5.

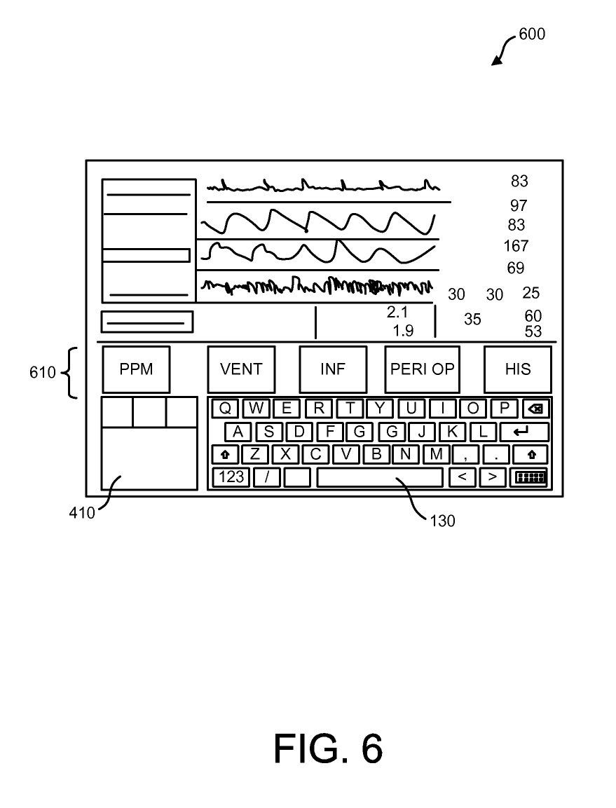

In some variations, as illustrated in diagram 600 of FIG. 6, the display can include a view having a region 610 within the graphical user interfaces where sub menus for specific patient data can be called up by pressing on a boxed or otherwise outlined region. These submenus can provide detailed information that is driven by specific clinical applications and can include advanced features for PPM (Physiological Patient Monitoring), VENT (ventilation), INF (Infusion Pumps), PERI OP (Perio Operative—Anesthesia, Medication, Pharma, etc.), and the like. These fields can be configured by the hospital or users according to care area needs. This region 610 can be displayed concurrently with the interactive keyboard 130 and, in some variations, also with the touchpad interface 410. It will be appreciated in some variations, the touchpad interface 410 alone can be provided in the display based on detected movement by the position sensor 114.

The position sensor 114 generates signals indicative of motion which can be used by the display 110 to determine whether or not to change the view to either include or exclude an interactive keyboard 130. For example, a rule can be defined such that if the front face of the display 110 is tilted beyond a predefined angle range (e.g., 30 degrees, etc.) from vertical, the view on the display causes the interactive keyboard 130 to be included (such as in FIG. 3). Conversely, if front face of the display 110 is tilted within a predefined angle range from vertical (e.g. 30 degrees, etc.), the view on the display can remove the keyboard (such as in FIG. 2). Other rules can be defined for triggering when the keyboard is included in the view based on factors such as rotational angle, rate of acceleration and the like. In some variations, a predefined period of time such as three seconds must elapse before the keyboard is added/removed from a particular view (to avoid situations in which the display 110 is being moved).

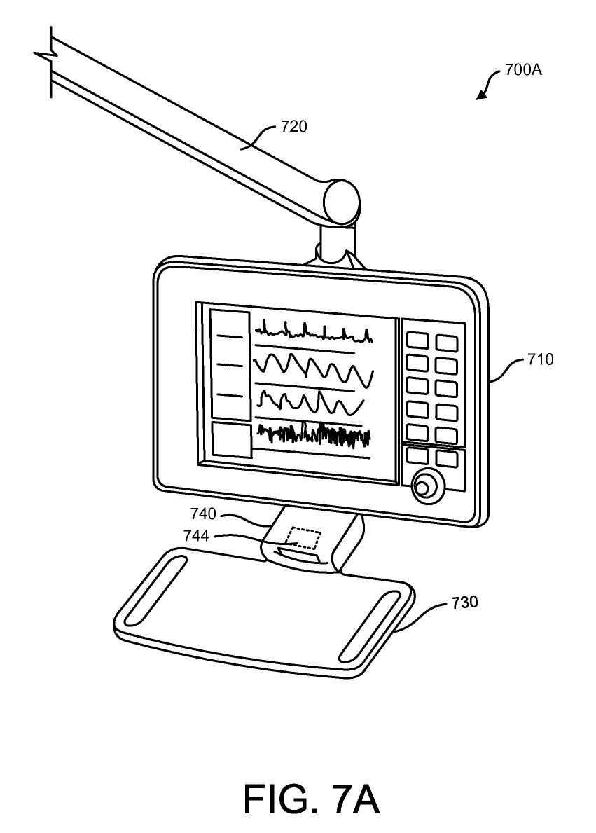

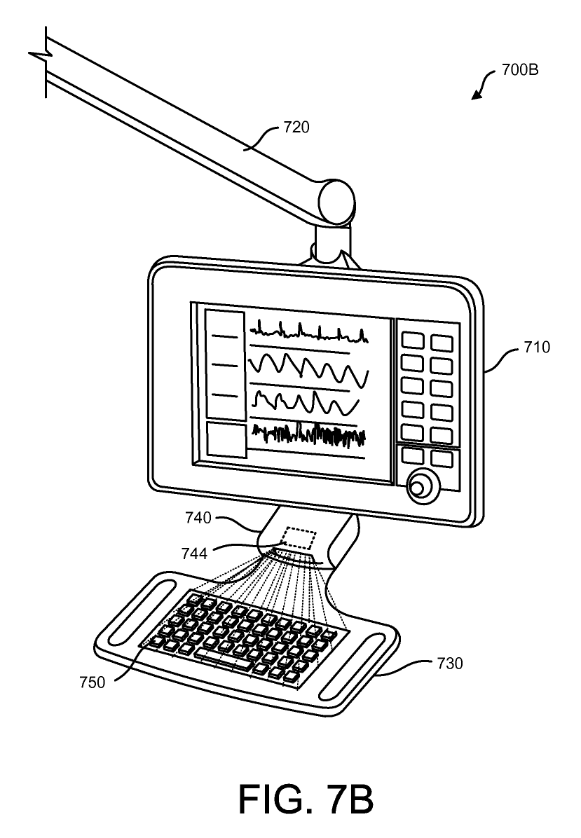

FIGS. 7A and 7B are diagrams 700A, 700B that illustrate a variation in which movement of an accessory 730 associated with a display 710 (as opposed to movement of the display 710) can cause a virtual keyboard 750 to be projected on a surface. The display 710 can, for example, include a mounting arm 720 affixed to a wall, ceiling, or other surface that can allow movement of the display 710 or which, in other variations, secures the position of the display 710. Various aspects as described above in connection with display 110 can also be applied to this implementation including having a position sensor 744 within the display 710 or the mounting arm 720.

In the example of FIG. 7B, the accessory 730 is a keyboard tray and the virtual keyboard 750 is projected on the tray. The accessory 730 can be coupled to a projection element 740 which, can for example, comprise a laser projection virtual keyboard 750 which can detect whether fingers of a user “activate” various projected keys. The projection element 740 can include one or more mounts that allow the accessory 730 to be selectively moved to stow the keyboard tray 730 (as in FIG. 7A) and in additionally in various positions for use (as in FIG. 7B).

The projection element 740 can include or otherwise be associated with a position sensor 744 (represented in dashed lines) that can detect when the accessory is moved from a first position (e.g., stowed position, etc.) to a second position (e.g., non-stowed position to enable use, etc.). This detected movement can be lateral, angular, or a combination of both. The dashed lines for position sensor 744 indicate that the position sensor can 744, in some variations, be a component internal to the projection element 740 (i.e., enclosed within a main housing of the projection element 740). The position sensor 744 can be at any position within or coupled to the projection element such that movement of the accessory 730 can be characterized or otherwise detected. In other variations, the position sensor 744 can be within the housing of the display 710. Further, while reference is made to a single position sensor 744 it can be appreciated that there can be two or more position sensors 744 that generate signals indicative of position (e.g., absolute position or orientation, relative movement or orientation, etc.) which can act in concert or be wholly separate. The position sensors 744 can take various forms including one or more of: mechanical switches, optical switches, accelerometers, angular position sensors, gyro sensors, linear position sensors, magnetic/Hall Effect sensors, and the like.

Once the position change has been detected as changing from the first position to the second position, the projection element 740 can project a virtual keyboard 750 onto the accessory 730. The projection element 740 in some cases is physically connected to the display 710 while in other arrangements the projection element 740 wirelessly communicates with the display 710 (either directly or indirectly) over protocols such as BLUETOOTH.

In some variations, the displays 110 and 710 can include a proximity sensor that can detect whether or not a user is near the display. This detection can in turn be used to determine whether or not to render the interactive keyboard 130 in the display 110 or to project the virtual keyboard on the accessory 730 (e.g., keyboard tray, etc.) at any given time. For example, the interactive keyboard 130 or the virtual keyboard 750 can be removed from the view or otherwise deactivated if the user walks away from the display 110, 710 as determined by the proximity sensor. In another example, the proximity sensor can be used to initiate rendering of the interactive keyboard 130 in the display 110 or to initiate the projection of the virtual keyboard 750 on the accessory 730 without the detection of motion or a change in orientation by the position sensor 114, 744. Stated differently, in some variations, the display 110, 710 can include or otherwise have a proximity sensor which, on its own, is used to render an interactive keyboard 130 in the display 110 or to project a virtual keyboard 750 on the accessory 430 associated with the display 710 when a user is within a predefined distance from the display 110, 710. Predefined distance in this regard may include a specific distance or detection by the proximity sensor that a user is proximal to the display 110, 710.

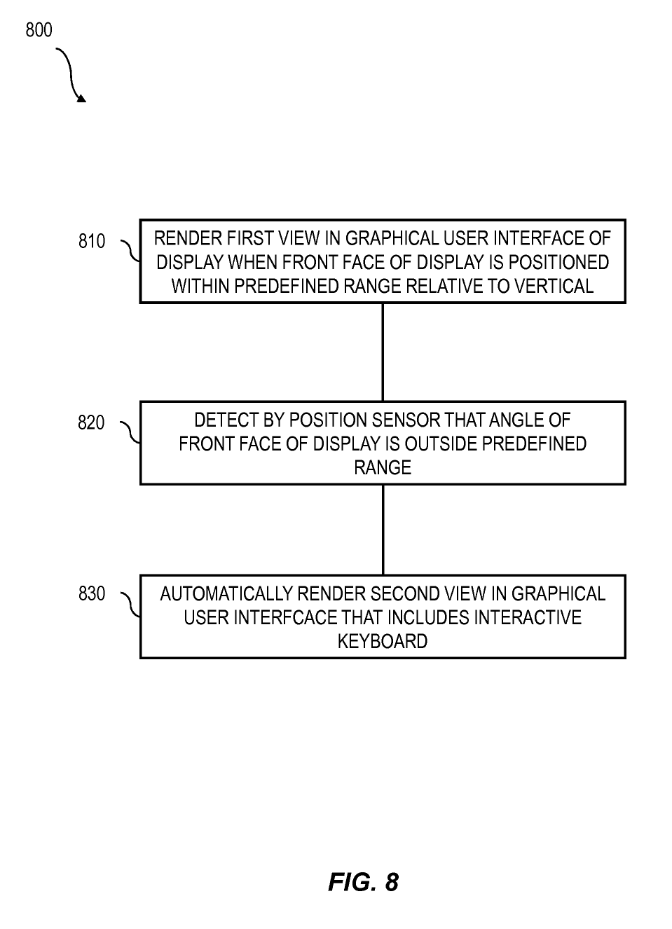

FIG. 8 is a process flow diagram 800 in which, at 810, a first view is rendered in a graphical user interface of a touch screen display when a front face of the display, on which the graphical user interface is rendered, is positioned within a predefined angle range relative to vertical. This first view does not include an interactive keyboard. Thereafter, at 820, a position sensor within or adjacent to the display detects that an angle of the front face of the display is beyond the predefined angle range. In response to the detecting, at 830, a second view is automatically rendered in the graphical user interface that includes an interactive keyboard.

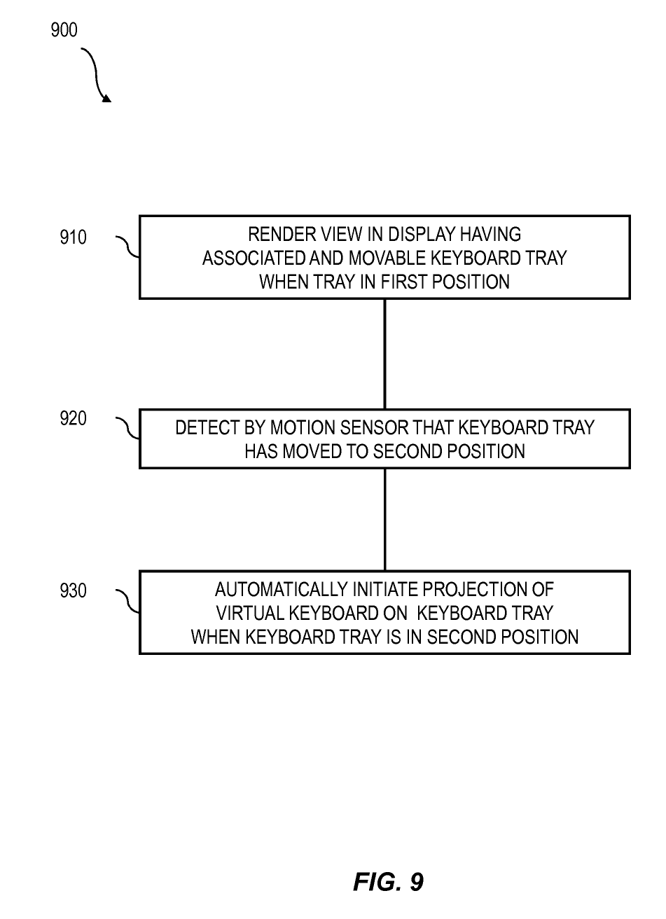

FIG. 9 is a process flow diagram 900 in which, at 910, a view is rendered in a graphical user interface of a display that has an associated and movable keyboard tray. Initially, the keyboard tray is in a first position. Thereafter, at 920, a position sensor within the keyboard tray detects a change in position of the keyboard tray relative to the first position beyond a predefined amount. As a result, at 930, projection of a virtual keyboard on a surface of the keyboard tray is automatically initiated in response to the detecting.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method comprising:
rendering, in a graphical user interface of a touch screen display, a first view when a front face of the display, on which the graphical user interface is rendered, is positioned within a predefined angle range relative to vertical, wherein the first view does not include an interactive keyboard;
detecting, by a position sensor, that an angle of the front face of the display is beyond the predefined angle range; and
automatically rendering, in the graphical user interface in response to the detecting, a second view that includes an interactive keyboard.

2. The method of claim 1, wherein at least one of the first view or the second view comprises patient medical data and the display forms part of a patient monitor.

3. The method of claim 1, wherein the detecting further comprises:
detecting that the angle of the front face of the display has exceeded the predefined angle range relative to vertical beyond a predefined amount of time.

4. The method of claim 1, wherein the second view encapsulates at least a portion of the first view.

5. The method of claim 1, wherein, in the second view, the interactive keyboard obscures at least a portion of the first view.

6. The method of claim 1, wherein the second view solely comprises the interactive keyboard.

7. The method of claim 1 wherein, in the second view, the keyboard does not obscure any portion of the first view.

8. The method of claim 1, wherein the display is secured to a first end of a mounting arm that is configured to be secured to a surface at a second end of the mounting arm.

9. The method of claim 8, wherein the position sensor is within the mounting arm.

10. The method of claim 8, wherein the mounting arm comprises a joint at the first end, wherein the position sensor detects relative motion between the joint and the display.

11. The method of claim 1, wherein the position sensor is within a housing of the display.

12. The method of claim 1, wherein the position sensor is selected from a group consisting of: an accelerometer, an angular position sensor, a gyro sensor, a linear position sensor, a magnetic/Hall Effect sensor, a mechanical switch, and an optical switch.

13. The method of claim 1 comprising:
determining, by a proximity sensor, that a user of the display is no longer within a predefined distance from the display; and
removing the interactive keyboard from the second view in response to the determining.

14. A method comprising:
rendering, in a graphical user interface of a display, a view in the display having an associated and movable keyboard tray when the keyboard tray is in a first position;
detecting, by a position sensor within the keyboard tray, a change in position of the keyboard tray relative to the first position beyond a predefined amount; and
automatically initiating projection of a virtual keyboard on a surface of the keyboard tray in response to the detecting.

15. The method of claim 14, wherein the change in position of the keyboard tray relative to the first position is based on at least one of lateral motion or angular motion of the keyboard tray.

16. The method of claim 14, wherein the first view comprises patient medical data and the display forms part of a patient monitor.

17. The method of any of claim 14, wherein the detecting comprises: detecting that an angle of the keyboard tray has exceeded a predefined angle range.

18. The method of claim 14, wherein the detecting comprises: detecting that a lateral position of the keyboard tray has exceeded a predefined distance relative to the first position.

19. The method of claim 14, wherein the detecting comprises:
detecting that the change in position from the first position has exceeded a predefined amount of time.

20. The method of claim 14, wherein the position sensor is selected from a group consisting of: an accelerometer, an angular position sensor, a gyro sensor, a linear position sensor, and a magnetic/Hall Effect sensor, a mechanical switch, and an optical switch.

21. The method of claim 14 further comprising:
determining, by a proximity sensor, that a user of the display is no longer within a predefined distance from the display; and
ceasing the projection of the virtual keyboard in response to the determining.

22. A method comprising:
rendering, in a graphical user interface of a touch screen display, a first view when a front face of the display, on which the graphical user interface is rendered, is positioned vertically or within a predefined angle range relative to vertical, wherein the first view does not include a user input area;
detecting, by at least one position sensor within or adjacent to the display, that an angle of the front face of the display is beyond the predefined angle range; and automatically rendering, in the graphical user interface in response to the detecting, a second view that includes a user input area.

23. The method of claim 22, wherein the user input area comprises at least one of: an interactive keyboard or a touchpad interface.

24. (canceled)

25. (canceled)

26. A system comprising:
a display comprising:
a housing;
at least one programmable data processor;
memory; and
a mounting arm comprising a position sensor that is secured, on a first end, to the housing, and is configured to be secured, on a second end, to a surface;
wherein the memory stores instructions which, when executed by the at least one programmable data processor, implement a method comprising:
rendering, in a graphical user interface of a touch screen display, a first view when a front face of the display, on which the graphical user interface is rendered, is positioned within a predefined angle range relative to vertical, wherein the first view does not include an interactive keyboard;
detecting, by a position sensor, that an angle of the front face of the display is beyond the predefined angle range; and
automatically rendering, in the graphical user interface in response to the detecting, a second view that includes an interactive keyboard.

27. The system of claim 26, wherein the mounting arm comprises a joint at the first end, wherein the position sensor detects relative motion between the joint and the display.
